(19) United States
(12) Patent Application Publication
(10) Pub. No.: US 2009/0247542 A1
(43) Pub. Date: Oct. 1, 2009

(54) SYNTHESES AND PREPARATIONS OF POLYMORPHS OF CRYSTALLINE ARIPIPRAZOLE

(75) Inventors: Monica Benito Velez, L'hospitalet De Llobregat (ES); Elies Molins I Grau, Sant Feliu de Llobregat (ES); Carmen Arnalot Aguilar, Girona (ES); Bernardino Mangion, Girona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona, ES (ES)

(21) Appl. No.: 11/911,623

(22) PCT Filed: Apr. 13, 2006
(86) PCT No.: PCT/IB2006/002301

§ 371 Date: Jul. 28, 2008

Related U.S. Application Data
(60) Provisional application No. 60/671,524, filed on Apr. 15, 2005.
(60) Provisional application No. 60/736,128, filed on Nov. 14, 2005.

(51) Int. Cl. A61K 31/496; C07D 401/12; A61P 25/18

(52) U.S. Cl. 514/253.07; 544/363

(57) ABSTRACT

The invention relates to polymorphic crystalline forms of aripiprazole, synthetic processes for their preparation and pharmaceutical compositions containing the same. These crystalline forms of aripiprazole can be readily milled and can be easily combined with various pharmaceutical adjuvants without effecting changes to their crystalline structure when, for example, pressed into tablet or capsule form.

### CROSS REFERENCE TO RELATED APPLICATIONS

This application claims priority to U.S. Provisional Application Nos. 60/671,524, filed Apr. 15, 2005 and 60/736,128, filed Nov. 14, 2005. These applications are expressly incorporated herein by reference in their entirety.

### BACKGROUND OF THE INVENTION

1. Field of the Invention

The invention relates to polymorphic crystalline forms of aripiprazole, synthetic processes for their preparation and pharmaceutical compositions containing the same. The crystalline forms of aripiprazole are stable, have reduced hygroscopicity and do not spontaneously convert to other polymorphic forms of aripiprazole. These crystalline forms of aripiprazole can be readily milled and can be easily combined with various pharmaceutical adjuvants without effecting changes to their crystalline structure when, for example, pressed into tablet or capsule form.

2. Discussion of the Related Art

Aripiprazole is the common name for 7-{4-[4-(2,3-dichlorophenyl)-1-piperazinyl]-butoxy}-3,4-dihydrocarbostyril or 7-{4-[4-(2,3-dichlorophenyl)-1-piperazinyl]-butoxy}-3,4-dihydro-2(1H)-quinolinone (Formula (I)).

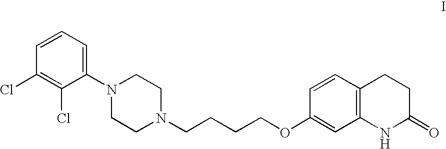

Aripiprazole is useful and approved for treating schizophrenia. In this regard, U.S. Pat. No. 5,006,528 discloses a process for the preparation and therapeutic use of aripiprazole.

As with many other physiologically active chemical compositions, aripiprazole shows polymorphism. Polymorphism is defined as the ability of a substance to crystallize in more than one crystal lattice arrangement. Polymorphism can influence many aspects of solid state properties of a drug. Thus, different crystal modifications of a substance (i.e., different polymorphs) may differ considerably from one another in their physical properties including, for example, their solubility characteristics, dissolution rates and bioavailability.

Aripiprazole has been approved by the FDA for the treatment of schizophrenia in 2, 5, 10, 15, 20 and 30 mg tablets for oral administration and is currently marketed under the brand name of Abilify®. Notably, in the Summary Basis of Approval (SBA) of New Drug Application 21-436, the innovator stated that “[t]he current tablet formulation of aripiprazole makes use of anhydrous Form I drug substance.” Aripiprazole was also approved in the United Kingdom for treating schizophrenia. The European Public Assessment Report for Abilify of the European Medicine Agency (EMEA) mentions the existence of polymorphs. In particular, the EMEA states that “[a]ripiprazole can exist in several crystalline forms, Form I was chosen for the development and commercialization.” According to the EMEA information, “[t]he formulation contains stable milled crystalline aripiprazole because of the limited solubility in water and the hydrophobic nature of the active substance.”

Until now, aripiprazole was known to exist in at least seven different crystalline forms. As discussed in WO 2003/026659, which is incorporated herein by reference in its entirety, seven known polymorphs of aripiprazole are (1) hydrate Form A, (2) anhydrous Form B, (3) anhydrous Form C, (4) anhydrous Form D, (5) anhydrous Form E, (6) anhydrous Form F and (7) anhydrous Form G. The various forms differ from each other in their physical and spectroscopic properties as well as in their methods of preparation.

According to WO 2003/026659, these various forms of aripiprazole can be prepared in the following ways:

Aripiprazole hydrate Form A has been obtained by milling conventional aripiprazole hydrate to a mean particle size of 50 μm or less.

Aripiprazole Form B has been prepared by several different processes including, for example: (a) drying aripiprazole hydrate Form A for 24 hours at 100° C. using a hot dryer, (b) drying aripiprazole hydrate Form A for 18 hours at 100° C. in a hot dryer and then heating for 3 hours at 120° C., (c) heating conventional hygroscopic aripiprazole anhydrous crystals or conventional aripiprazole hydrate at 100° C. or 120° C. for 3 to 50 hours.

Anhydrous Form C can be obtained by heating conventional anhydrous aripiprazole crystals at a temperature of about 145° C. to yield colorless prism crystals.

Anhydrous Form D is obtained by recrystallizing conventional anhydrous aripiprazole crystals from toluene to form colorless plate crystals.

Anhydrous Form E can be prepared by heating and dissolving conventional anhydrous aripiprazole crystals in acetonitrile and cooling the resulting product to form colorless needle crystals.

Anhydrous Form F can be obtained by heating a suspension of conventional anhydrous aripiprazole crystals in acetone to form colorless prism crystals.

Anhydrous Form G is obtained by maintaining a glassy state anhydrous aripiprazole in a sealed vessel at room temperature for approximately 6 months. The initial glassy state anhydrous aripiprazole can be obtained by heating and melting anhydrous aripiprazole crystals at 170° C.

WO 2004/083183 describes the preparation of two forms of aripiprazole, designated therein as Forms I and II. Form I is obtained by crystallization from acetone, ethyl acetate, methanol or ethanol. Form II is obtained by dissolving aripiprazole in tetrahydrofuran followed by vacuum drying at 25° C. or spray drying. A comparison of the data (e.g., the X-ray diffractograms) from WO 2004/083183 to the data in WO 2003/026659 suggests that Form I corresponds to anhydrous Form D and that Form II corresponds to the aripiprazole hydrate Form A.

WO 2004/106322 also describes the preparation of several forms of aripiprazole, designated therein as Forms II, III and IV. Form II is obtained by contacting or crystallizing the product from isopropyl alcohol, isopropyl acetate, methanol or mixtures thereof. Form III is obtained by contacting or crystallizing the product from isobutyl acetate or ethanol, and Form IV is obtained by contacting or crystallizing the product from acetone, t-butyl alcohol and/or mixtures thereof or heating aripiprazole to about 150° C. A comparison of the data (e.g., the X-ray diffractograms) from WO 2004/106322 to the data in WO 2003/026659 suggests that Form II corresponds to the anhydrous Form D and that Form IV corresponds to Form C.

WO 2005/009990 describes the preparation of a crystalline non-hygroscopic form of aripiprazole designated therein as Form III and two novel solvates designated as aripiprazole methanolate Form IV and aripiprazole ethylene dichloride Form V. Form III is obtained by crystallization from a mixture of methyl t-butyl ether, acetonitrile and tetrahydrofuran. Aripiprazole methanolate Form IV is obtained by crystallization from a mixture of methanol and tetrahydrofuran. Aripiprazole ethylene dichloride Form V is obtained by crystallization from ethylene dichloride. Comparison of the X-ray diffractogram of Form III from WO 2005/009990 to the above-described forms indicates it corresponds to the anhydrous Form D of WO 2003/026659.

During the Proceedings of the 4th Japan-Korean Symposium on Separation Technology (Oct. 6-8, 1996), it was disclosed that aripiprazole has two types of anhydrous polymorphs (type 1 and type 2) and a hydrous crystal (type 3). The anhydrous type 1 crystals of aripiprazole could be prepared by recrystallizing aripiprazole from an ethanol solution or by heating hydrous crystal type 3 at 80° C. Anhydrous crystal type 1 has a melting point of 140° C. The anhydrous type 2 crystals of aripiprazole could be prepared by heating anhydrous type 1 crystals of aripiprazole to 130 to 140° C. for 15 hours. The melting point of anhydrous type 2 crystals was 150° C. When anhydrous type 1 and 2 crystals of aripiprazole were recrystallized from an alcoholic solvent containing up to 20% (v/v) water, the crystals were converted to hydrous crystals type 3. WO 2005/058835 also describes anhydrous aripiprazole type 2, designated therein as Form II and herein as Form J, and methods of preparing it.

According to U.S. Pat. No. 5,006,528, and as demonstrated in Scheme 1 (below), aripiprazole can be prepared by condensing 7-(4-bromobutoxy)-3,4-dihydro-2(1H)-quinolinone (i.e., Compound II; 7-(4-bromobutoxy)-3,4-dihydrocarbostyril) with 1-(2,3-dichlorophenyl)piperazine (i.e., Compound III) in acetonitrile under basic conditions (e.g., triethylamine) and in the presence of sodium iodide at reflux temperature. After removal of the solvent by evaporation, the resulting residue can be dissolved in chloroform, washed with water and dried with anhydrous magnesium sulphate. After removal of the solvent by evaporation, the residue can be recrystallized (twice) from ethanol to yield colorless flake crystals having a melting point of 139-139.5° C.

As demonstrated in Scheme 1 (below), WO 2003/026659 and WO 2004/063162 each describe an alternative process for obtaining aripiprazole generally comprising condensing 7-(4-chlorobutoxy)-3,4-dihydro-2(1H)-quinolinone (i.e., Compound IV; 7-(4-chlorobutoxy)-3,4-dihydrocarbostyril) and 1-(2,3-dichlorophenyl)piperazine (i.e., Compound III) in the form of its hydrochloride salt in water and in the presence of potassium carbonate. Crude crystals of aripiprazole are then filtered from the reaction media and recrystallized from anhydrous ethyl acetate by means of azeotropic distillation.

According to WO 2003/026659, the resulting crystals from ethyl acetate are dried for 14 hours at 60° C. and then recrystallized from ethanol. The resulting crystals are then dried for 40 hours at 80° C. to obtain anhydrous crystals of aripiprazole having a melting point of 140° C.

Alternatively, WO 2004/063162 describes drying the obtained crystals (from ethyl acetate) under reduced pressure (˜50 torr) at 50° to 60° C. for 3 hours to obtain aripiprazole having a melting point of 140° C.

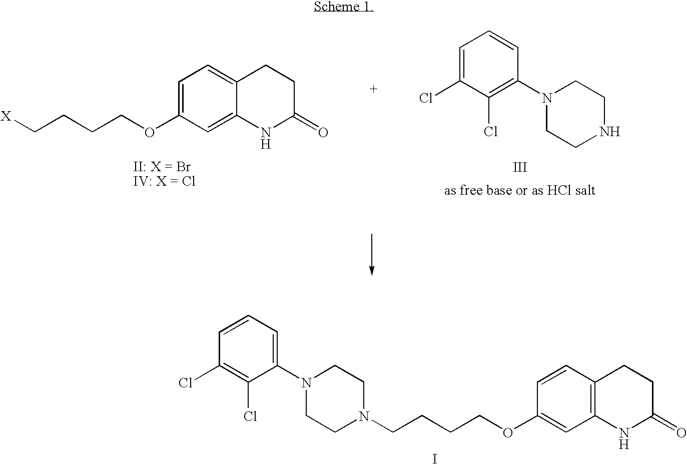

No data regarding the ethanol content of aripiprazole obtained from ethanol has been reported in the literature. It has been observed here, however, that different polymorphic crystalline forms of aripiprazole and/or mixtures thereof can be obtained depending on the drying temperature of the product obtained from ethanol.

Additionally, it has been observed that an ethanol solvate of aripiprazole (recrystallized from ethanol) can be obtained by drying the obtained product for 6 hours at room temperature, 40° C. or 60° C. This aripiprazole hemiethanolate, designated herein as Form H, contains approximately 5% of residual ethanol and has an XRD diffractogram as depicted in FIG. 2.

It has also been observed that aripiprazole hemiethanolate Form H can be converted to an anhydrous aripiprazole, designated herein as Form L (showing an XRD diffractogram as depicted in FIG. 3) or into a mixture of Forms L and H (showing an XRD diffractogram as depicted in FIG. 14) depending on the drying temperature and conditions.

### SUMMARY OF THE INVENTION

The invention comprises-polymorphic forms of aripiprazole (designated herein as aripiprazole Forms J and L), methods of making the same and formulations of the same. These forms of aripiprazole can be prepared by a novel process of recrystallizing aripiprazole (which has been prepared, for example, according to the herein described literature procedures) from ethanol or a ketonic solvent. Preferred ketonic solvents include, for example, acetone and methyl ethyl ketone (most preferred).

The invention further includes formulations and pharmaceutical compositions containing these crystalline forms of aripiprazole (i.e., comprising aripiprazole Forms J and/or L) generally comprising obtaining aripiprazole by recrystallization from methyl ethyl ketone (or other ketonic solvent(s)) or ethanol, respectively, in which aripiprazole and the chosen solvent are loaded into a suitable reactor and stirred in suspension or solution at reflux temperature and then cooled and filtered. Using this general method, which can also include using other ketonic solvents (e.g., acetone, mixtures of acetone and methyl ethyl ketone, etc.), avoids the use of other less desirable/more expensive solvents and limits the incremental increase in solvent volumes, thus increasing reactor productivity relative to the methods described in the literature.

The process for preparing aripiprazole Form J comprises the steps of suspending/dissolving aripiprazole in a ketonic solvent (e.g., acetone, methyl ethyl ketone, mixtures thereof), refluxing the solution for approximately 30 minutes to 1 hour, cooling the solution to approximately 0° C. to 5° C., maintaining the solution for approximately 2 to 4 hours at approximately 0° C. to 5° C. and isolating the resulting solid by filtration. The process can be repeated as necessary.

The process for preparing aripiprazole Form L comprises the steps of combining aripiprazole and ethanol, refluxing the suspension until dissolution, cooling the solution to approximately 0° C. to 5° C., maintaining the solution at this temperature for approximately 30 minutes to 2 hours and isolating the resulting solid by filtration. The process can be repeated as necessary. Drying the obtained product for 6 hours at room temperature, 40° C. or 60° C. yields aripiprazole hemiethanolate Form H. Drying the obtained product for approximately 6 hours at approximately 60-120° C. yields aripiprazole Form L.

Additional steps can include treating the solution with a decolorizing agent to improve the color and appearance of the resulting crystals and/or additional filtration steps to remove impurities (e.g., insolubles). The decolorizing agent can be any conventional decolorizing agent, including, for example, alumina, activated alumina, silica and charcoal. Both the addition of the decolorizing agent and/or any additional filtration steps can be conducted at a temperature preferably between room temperature and below the reflux temperature of the ketone solvent, preferably below 70° C.

The invention further includes formulating aripiprazole Forms J and/or L into readily usable dosage units for the therapeutic treatment (including prophylactic treatment) of mammals including humans. Such formulations may include, among other things, various pharmaceutical carriers and/or diluents.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the X-ray powder diffractogram of aripiprazole Form J obtained in Example 1;

FIG. 2 illustrates the X-ray powder diffractogram of aripiprazole Form H obtained in Reference Example 1;

FIG. 3 illustrates the X-ray powder diffractogram of aripiprazole Form L obtained in Comparative Example 1C;

FIG. 4 illustrates the X-ray powder diffractogram of aripiprazole Form K obtained in Reference Example 2;

FIG. 5 illustrates the Differential Scanning Calorimetry (DSC) thermogram in an open pan of aripiprazole Form J obtained in Example 1;

FIG. 6 illustrates the Differential Scanning Calorimetry (DSC) thermogram in an open pan of aripiprazole Form H obtained in Reference Example 1;

FIG. 7 illustrates the Differential Scanning Calorimetry (DSC) thermogram in a sealed pan of aripiprazole Form H obtained in Reference Example 1;

FIG. 8 illustrates the Differential Scanning Calorimetry (DSC) thermogram in an open pan of aripiprazole Form L obtained in Comparative Example 1C;

FIG. 9 illustrates the Differential Scanning Calorimetry (DSC) thermogram in an open pan of aripiprazole Form K obtained in Reference Example 2;

FIG. 10 illustrates the Thermogravimetric Analysis (TG) thermogram of aripiprazole Form H obtained in Reference Example 1;

FIG. 11 illustrates the Infrared (IR) spectra of aripiprazole Form J obtained in Example 1;

FIG. 12 illustrates the Infrared (IR) spectra of aripiprazole Form H obtained in Reference Example 1;

FIG. 13 illustrates the Infrared (IR) spectra of aripiprazole Form L obtained in Comparative Example 1C;

FIG. 14 illustrates the X-ray powder diffractogram of aripiprazole obtained in Comparative Example 6A corresponding to a mixture of aripiprazole Form L and Form H; and

FIG. 15 illustrates the molecular structure of aripiprazole Form J with the atom-labelling scheme.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention comprises polymorphic forms of aripiprazole (designated herein as aripiprazole Forms J and L), methods of making the same and formulations of the same.

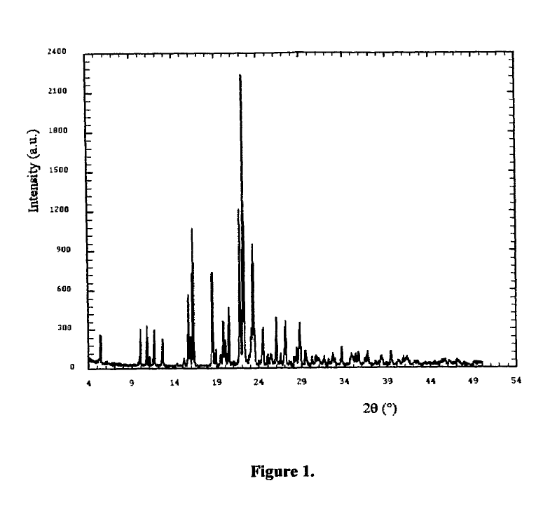

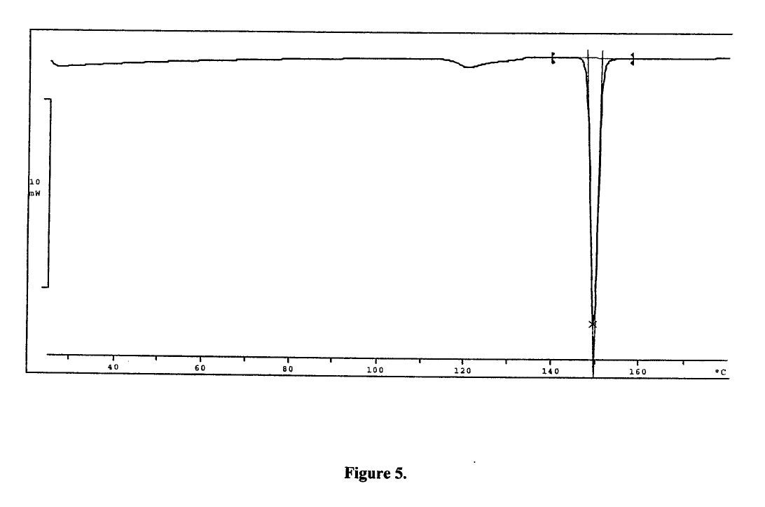

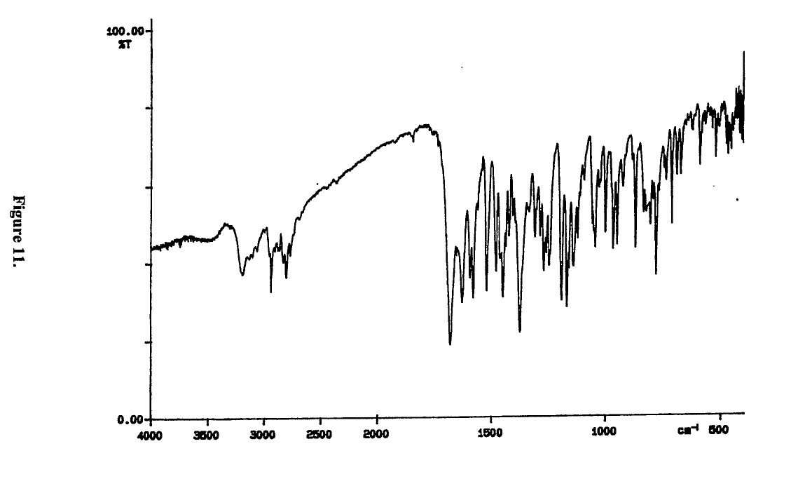

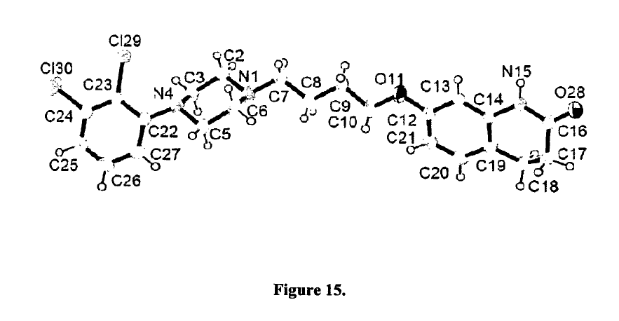

Aripiprazole Form J made according to the processes of the invention is characterized by having a melting point range of approximately 147.5-149.5° C. FIG. 1 illustrates the X-ray powder diffraction pattern (2θ) (±0.2°) of aripiprazole Form J, which has its main peaks at 5.33°, 9.93°, 10.71°, 11.55°, 12.55°, 15.64°, 15.90°, 16.23°, 18.49°, 18.89°, 19.45°, 19.75°, 19.99°, 20.42°, 21.77°, 22.22°, 23.27°, 24.43°, 25.97°, 27.04°, 28.36°, 28.73°, 29.42° and 33.61°. FIG. 5 illustrates the differential scanning calorimetry (open pan) of aripiprazole Form J, which exhibits two endothermic peaks at approximately 120° C. and approximately 149° C. FIG. 11 illustrates the infrared spectrum of aripiprazole Form J, which has its main peaks at 3192, 2939, 2831, 2805, 2768, 1680, 1628, 1593, 1579, 1521, 1479, 1450, 1437, 1423, 1375, 1309, 1285, 1270, 1260, 1247, 1192, 1169, 1160, 1142, 1122, 1045, 1000, 966, 949, 869, 805, 781 and 712 cm⁻¹. Aripiprazole Form J made according to the processes of the invention is further characterized by having low hygroscopicity (i.e., a moisture content of 0.5% or less, preferably less than 0.3% after storing anhydrous aripiprazole Form J for 24 hours at a temperature of 60° C. and a humidity level of 100%), a high purity (>99.8% according to HPLC), a low residual solvent content and is generally free of insoluble materials/compounds. The prepared aripiprazole Form J also has a mean particle size of about 100 μm or less, preferably about 50 μm or less and more preferably about 30 μm or less. FIG. 15 illustrates the molecular structure of aripiprazole Form J with the atom-labelling scheme. The basic crystallographic data for single crystal of aripiprazole Form J is as follows:

| | |
|---|---|
| Crystal Size: | 2.30 × 0.90 × 0.40 mm³ |
| Crystal System, space group | Monoclinic, P 2₁ |
| Unit Cell dimensions | a = 8.874(2) \acute{Å} |
| | b = 7.766(4) \acute{Å} |
| | c = 16.4808(14) \acute{Å} |
| | β = 93.136(12) ° |
| | α = γ = 90° |
| Volume | 1134.1(6) \acute{Å}³ |
| Z | 2 |
| Calculated density | 1.313 mg/m³ |

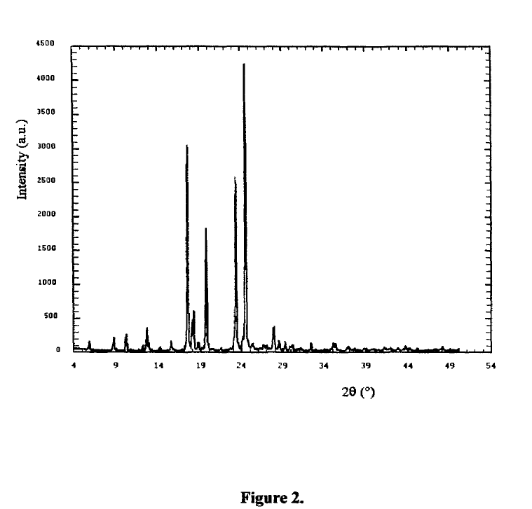

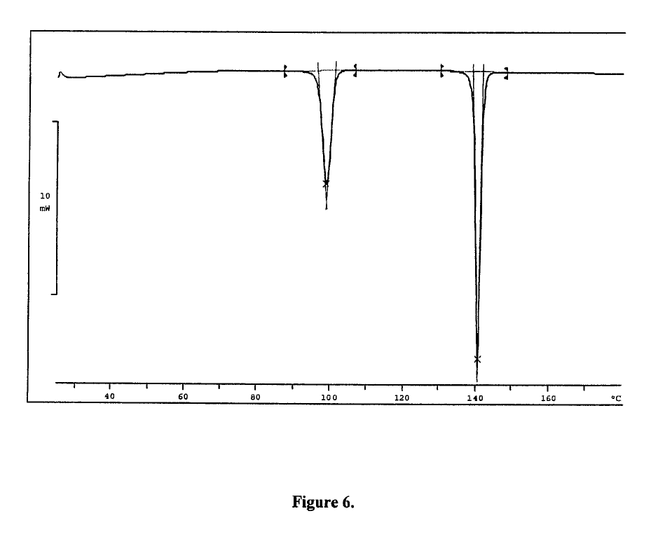

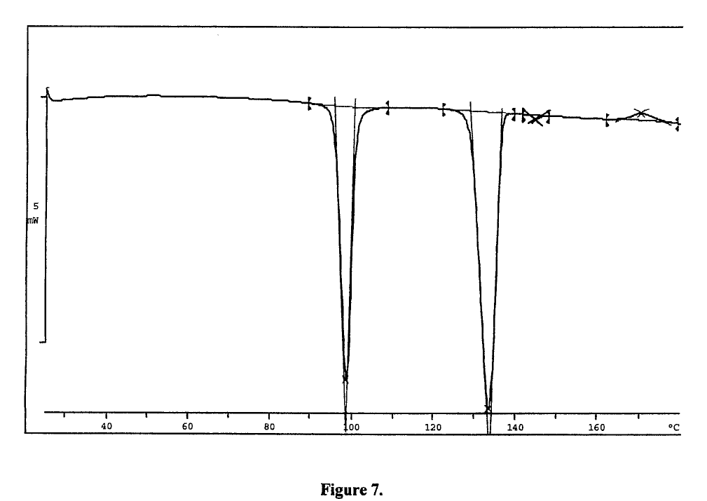

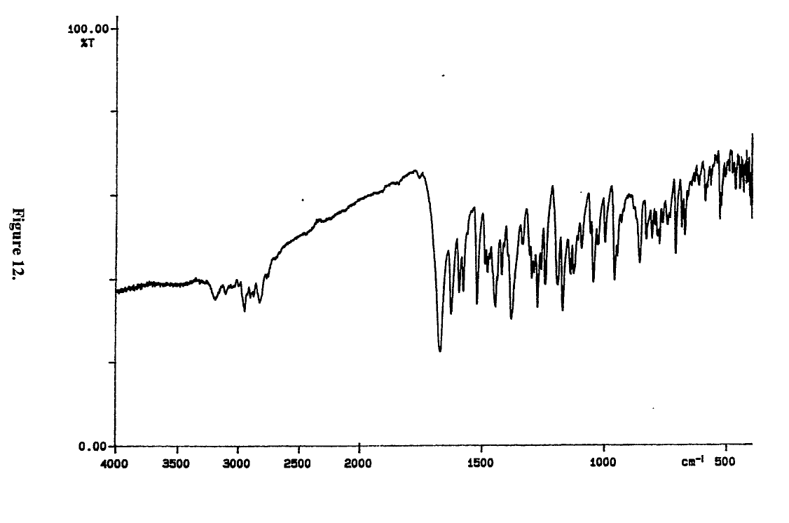

Aripiprazole Form H is characterized by being an ethanol solvate, particularly an hemiethanolate solvate, containing approximately 5% of residual ethanol. This is in accordance with the assay result (˜95%) and TG loss of weight (˜4.8%). FIG. 2 illustrates the X-ray powder diffraction pattern (2θ) (±0.2°) of aripiprazole Form H which has its main peaks at 10.2°, 12.7°, 17.4°, 18.0°, 18.7°, 19.7°, 23.3°, 24.4°, 27.8° and 28.5°. FIG. 6 illustrates the differential scanning calorimetry (open pan) of aripiprazole Form H, which exhibits two endothermic peaks at approximately 99° C. and approximately 140° C. FIG. 7 illustrates the differential scanning calorimetry (sealed pan) of aripiprazole Form H, which exhibits two endothermic peaks at approximately 99° C. and approximately 133° C. FIG. 12 illustrates the infrared spectrum of aripiprazole Form H which has its main peaks at 3483, 3065, 2949, 2886, 2823, 1780, 1625, 1594, 1578, 1398, 1327, 1305, 1267, 1242, 1189, 1114, 1096, 1061, 1045, 947, 933, 845, 786, 718 and 588 cm⁻¹. Aripiprazole Form H is further characterized by high purity (according to HPLC) and is generally free of insoluble materials/compounds. The prepared aripiprazole Form H also has a mean particle size of about 100 μm or less, preferably about 50 μm or less and more preferably about 30 μm or less.

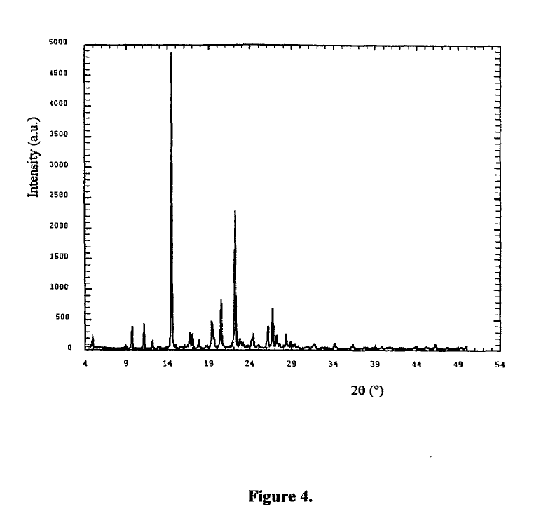

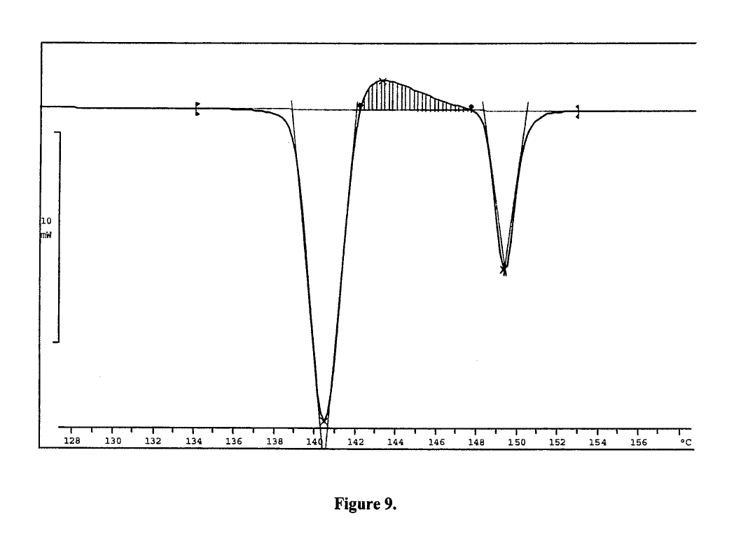

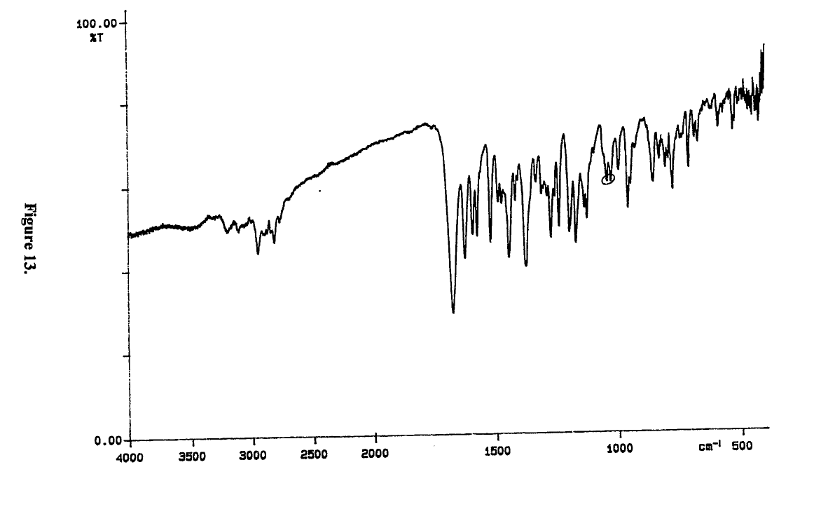

Aripiprazole Form K is characterized by having a melting point of approximately 150° C. FIG. 4 illustrates the X-ray powder diffraction pattern (2θ) (±0.2°) of aripiprazole Form K which has its main peaks at 11.0°, 12.2°, 14.3°, 14.5°, 16.6°, 17.0°, 19.4°, 19.5°, 20.3°, 20.5°, 22.1°, 22.8°, 24.3°, 26.0°, 26.6°, 27.1° and 28.3°. FIG. 9 illustrates the differential scanning calorimetry (open pan) of aripiprazole Form K, which exhibits two endothermic peaks at approximately 140° C. and approximately 149° C. The infrared spectrum of aripiprazole Form K, which has its main peaks at 3102, 2945, 2811, 2769, 1676, 1628, 1593, 1576, 1411, 1335, 1290, 1274, 1258, 1240, 1199, 1030, 795, 778, 744 and 570 cm⁻¹, is substantially equivalent to the IR spectra of aripiprazole Form L shown in FIG. 13. Aripiprazole Form K is further characterized by high purity (99.8% according to HPLC), a low residual solvent content and is generally free of insoluble materials/compounds. The prepared aripiprazole Form K also has a mean particle size of about 100 μm or less, preferably about 50 μm or less and more preferably about 30 μm or less.

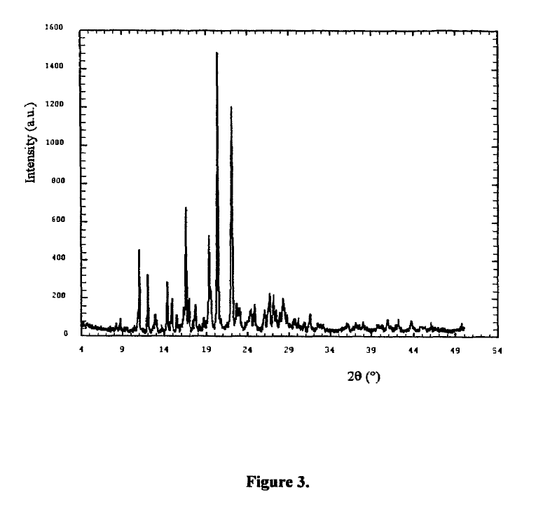

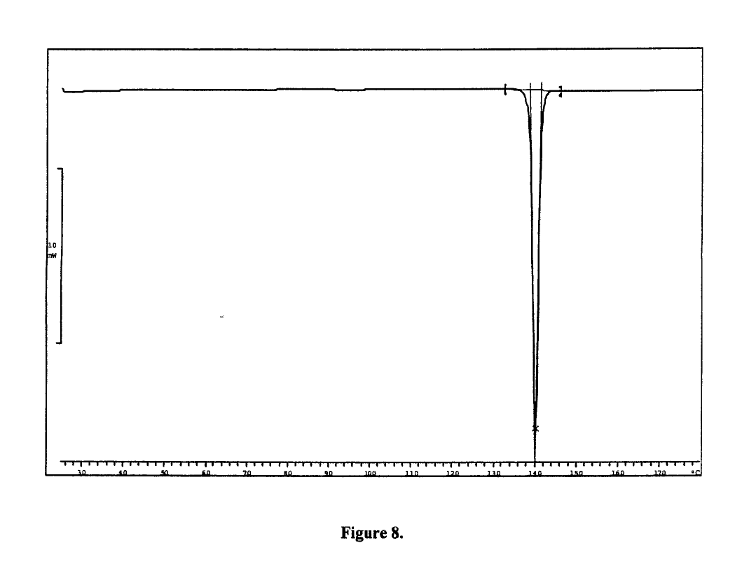

Aripiprazole Form L made according to the processes of the invention is characterized by having a melting point range of approximately 138.8-139.3° C. FIG. 3 illustrates the X-ray powder diffraction pattern (2θ) (±0.2° of aripiprazole Form L, which has its main peaks at 11.0°, 12.1°, 14.4°, 14.9°, 16.6°, 17.0°, 19.3°, 19.5°, 20.4°, 22.1°, 26.6°, 27.1° and 28.3°. FIG. 8 illustrates the differential scanning calorimetry (open pan) of aripiprazole Form L, which exhibits an endothermic peak at approximately 139° C. FIG. 13 illustrates the infrared spectrum of aripiprazole Form L, which has its main peaks at which has its main peaks at 3102, 2945, 2811, 2769, 1676, 1628, 1593, 1576, 1411, 1335, 1290, 1274, 1258, 1240, 1199, 1030, 795, 778, 744 and 570 cm⁻¹. Aripiprazole Form L made according to the processes of the invention is further characterized by high purity (>99.8% according to HPLC).

The invention further includes a process for preparing aripiprazole Form J generally comprising obtaining aripiprazole Form J by recrystallization from a ketonic solvent (e.g., acetone, methyl ethyl ketone) in which the aripiprazole and ketonic solvent are placed in a suitable reactor and stirred in suspension at reflux temperature and then cooled and filtered. This process may be repeated several times to further purify the aripiprazole Form J. Preferred ketonic solvents include, for example, acetone and methyl ethyl ketone (most preferred). The initial aripiprazole can be obtained by any available or known method including, for example, those discussed herein. Additional steps can include adding decolorizing agents and/or performing additional filtration steps.

The invention further includes a process for preparing aripiprazole Form L generally comprising drying an aripiprazole alcoholic solvate. Preferred alcoholic solvate include, for example, methanol solvate, ethanol solvate, propanol solvate and butanol solvate. A more preferred alcoholic solvate include an ethanol solvate. The most preferred ethanol solvate is an hemiethanolate. The initial aripiprazole solvate can be obtained by any available or known method including, for example, those discussed herein.

For example, aripiprazole hemiethanolate Form H can be obtained by recrystallization from an alcoholic solvent in which the aripiprazole and alcoholic solvent are placed in a suitable reactor and stirred in suspension at reflux temperature and then cooled and filtered. This process may be repeated several times to further purify the aripiprazole Form H. The obtained product is dried at temperatures ranging from room temperature to 60° C. for approximately 6 hours. Preferred alcoholic solvents include, for example, methanol, ethanol, propanol and butanol. The most preferred alcoholic solvent is ethanol. The initial aripiprazole can be obtained by any available or known method including, for example, those discussed herein. Additional steps can include adding decolorizing agents and/or performing additional filtration steps.

Similarly, aripiprazole Form K can be obtained by recrystallization from an ester solvent in which the aripiprazole and ester solvent are placed in a suitable reactor and stirred in suspension at reflux temperature and then cooled and filtered. This process may be repeated several times to further purify the aripiprazole Form K. Preferred ester solvents include, for example, ethyl acetate, propyl acetate, butyl acetate. The most preferred ester solvent is ethyl acetate. The initial aripiprazole can be obtained by any available or known method including, for example, those discussed herein. Additional steps can include adding decolorizing agents and/or performing additional filtration steps.

The invention further includes pharmaceutically acceptable salts of aripiprazole and a process for making the same. Pharmaceutically acceptable salts can be readily prepared by conventional techniques. A “pharmaceutically acceptable salt” is a salt that retains the biological effectiveness of the free acids and bases of the specified compound and that is not biologically or otherwise undesirable. Examples of pharmaceutically acceptable salts include those salts prepared by reaction of aripiprazole Form H, Form J, K and/or Form L with a mineral or organic acid, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyn-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, pheylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates.

The desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alphahydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

The invention further includes formulating aripiprazole Forms J and/or aripiprazole Form L, either alone or in combination with other crystalline forms or aripiprazole and pharmaceutically acceptable salts thereof into readily usable dosage units for the therapeutic treatment (including prophylactic treatment) of mammals including humans. Such formulations are normally formulated in accordance with standard pharmaceutical practice as a pharmaceutical composition. According to this aspect of the invention there is provided a pharmaceutical composition that comprises aripiprazole Form J alone, aripiprazole Form L alone, or in combination with one another or other crystalline forms of aripiprazole and pharmaceutically acceptable salts thereof in association with a pharmaceutically acceptable diluent or carrier.

The compositions of the invention may be in a form suitable for oral use (for example as tablets, fast-dissolving tablets, lozenges, hard or soft capsules, aqueous or oily suspensions, emulsions, dispersible powders or granules, syrups or elixirs). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate, calcium carbonate and different types of cellulose such as powdered cellulose or microcrystalline cellulose; granulating and disintegrating agents such as corn starch and its derivatives, crospovidone, croscarmellose and/or algenic acid; binding agents such as starch and pregelatinized starch; lubricating agents such as magnesium stearate, stearic acid or talc; preservative agents such as sodium benzoate, ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate, kaolin or cellulose, a disintegrating agent such as corn starch and its derivatives, crospovidone and croscarmellose, or as soft gelatin capsules in which the active ingredient is mixed with water or an oil such as peanut oil, liquid paraffin, olive oil or glyceryl oleate derivatives.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as the sodium salt of benzoic acid, ethyl or propyl p-hydroxybenzoate), anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the Form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soybean, lecithin, and esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The amount of a compound of this invention that is combined with one or more excipients to produce a single dosage form will necessarily vary depending upon the host treated and the particular route of administration. For example, a formulation intended for oral administration to humans may contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

Specific examples of suitable pharmaceutical compositions containing aripiprazole appear in Tables I-IV below (all units expressed in milligrams).

Table I illustrates a representative pharmaceutical composition (wet granulation) containing aripiprazole.

TABLE I

| | 30 mg | 15 mg | 10 mg | 5 mg |
|---|---|---|---|---|
| | Dosage | Dosage | Dosage | Dosage |
| Aripiprazole | 30 | 15 | 10 | 5 |
| Iron Oxide Red | 0.06 | — | 0.02 | — |
| Indigo Carmine Aluminum Lake | — | — | — | 0.2 |
| Iron Oxide Yellow | — | 0.2 | — | — |
| Magnesium Estereate | 2.4 | 0.8 | 0.8 | 0.8 |
| Avicel PH101 | 27.15 | 9.05 | 9.05 | 9.05 |
| L-HPC | 6 | 2 | 2 | 2 |
| Lactose Monohydrate | 186.54 | 57 | 62.18 | 67 |
| Pregelatinized Starch | 18 | 6 | 6 | 6 |
| Aerosil 200 | 2.85 | 0.95 | 0.95 | 0.95 |
| Croscarmellose Sodium | 12 | 4 | 4 | 4 |
| Total weight comp | 285 | 95 | 95 | 95 |

Table II illustrates a representative pharmaceutical composition (direct compression) containing aripiprazole.

TABLE II

| | 30 mg | 15 mg | 10 mg | 5 mg |
|---|---|---|---|---|
| | Dosage | Dosage | Dosage | Dosage |
| Aripiprazole | 30 | 15 | 10 | 5 |
| Iron Oxide Red | 0.06 | — | 0.02 | — |
| Indigo Carmine Aluminum Lake | — | — | — | 0.2 |
| Iron Oxide Yellow | — | 0.2 | — | — |
| Magnesium Estereate | 2.85 | 0.95 | 0.95 | 0.95 |
| Avicel PH200 | 99.28 | 33.09 | 33.09 | 33.09 |
| Lactose | 110.06 | 31.51 | 36.69 | 41.51 |
| Pregelatinized Starch | 39.9 | 13.3 | 13.3 | 13.3 |
| Aerosil 200 | 2.85 | 0.95 | 0.95 | 0.95 |
| Total weight comp | 285 | 95 | 95 | 95 |

Table III illustrates a representative pharmaceutical composition (aqueous granulation) containing aripiprazole.

TABLE III

| | 30 mg | 15 mg | 10 mg | 5 mg |
|---|---|---|---|---|
| | Dosage | Dosage | Dosage | Dosage |
| Aripiprazole | 30 | 15 | 10 | 5 |
| Iron Oxide Red | 0.06 | — | 0.02 | — |
| Indigo Carmine Aluminum Lake | — | — | — | 0.2 |
| Iron Oxide Yellow | — | 0.2 | — | — |
| Magnesium Estereate | 2.85 | 0.95 | 0.95 | 0.95 |
| Avicel PH102 | 99.28 | 33.09 | 33.09 | 33.09 |
| Lactose | 132.86 | 39.11 | 44.29 | 49.11 |
| PVP K25 | 14.25 | 4.75 | 4.75 | 4.75 |
| Crospovidone | 5.7 | 1.9 | 1.9 | 1.9 |
| Total weight comp | 285 | 95 | 95 | 95 |

Table IV illustrates a representative pharmaceutical composition (wet granulation) containing aripiprazole.

TABLE IV

| | 30 mg | 15 mg | 10 mg | 5 mg |
|---|---|---|---|---|
| | Dosage | Dosage | Dosage | Dosage |
| Aripiprazole | 30 | 15 | 10 | 5 |
| Iron Oxide Red | 0.06 | — | 0.02 | — |
| Indigo Carmine Aluminum Lake | — | — | — | 0.2 |
| Iron Oxide Yellow | — | 0.2 | — | — |
| Magnesium Stereate | 2.4 | 0.8 | 0.8 | 0.8 |
| Lactose Monohydrate | 186.54 | 57 | 62.18 | 67 |
| Microcrystalline Cellulose | 30 | 10 | 10 | 10 |
| Hydroxylpropyl Cellulose | 6 | 2 | 2 | 2 |
| Maize Starch | 30 | 10 | 10 | 10 |
| Total weight comp | 285 | 95 | 95 | 95 |

The representative pharmaceutical compositions described in Table IV were prepared by mixing a portion of the lactose monohydrate with the active pharmaceutical ingredient aripiprazole in a suitable blender. The blend was dried for two hours in a fluid bed while keeping the product temperature at 70° C.+/−5° C. After drying, the remaining portion of the lactose monohydrate, the red iron oxide, the microcrystalline cellulose and the maize starch were sieved, added to the previous blend and mixed. The obtained blend was then granulated using an aqueous hydroxypropyl cellulose solution. The obtained granules were dried and sieved through a 1 mm size mesh and then blended with the magnesium stearate. The resulting ready to press blend was compressed in a rotary tableting machine into suitable sized tablets containing 5, 10, 15 or 30 mg of aripiprazole.

The size of the dose for therapeutic or prophylactic purposes of the compounds of the invention will naturally vary according to the nature and severity of the conditions, the age and sex of the animal or patient and the route of administration, according to well known principles of medicine. For example, the method may comprise at least one of an hourly administration, a daily administration, a weekly administration, or a monthly administration of one or more compositions described herein.

According to the present invention, suitable methods of administering the therapeutic composition of the present invention to a patient include any route of in vivo administration that is suitable for delivering the composition into a patient. The preferred routes of administration will be apparent to those of skill in the art, depending on the type of condition to be prevented or treated, and/or the target cell population.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

### SPECIFIC EXAMPLES

The following examples are for illustrative purposes only and are not intended, nor should they be interpreted to, limit the scope of the invention.

General Experimental Conditions:

i. Infrared Spectra

Fourier transform infrared spectra were acquired on a Perkin-Elmer 1600 series FTIR spectrometer and polymorphs were characterized in potassium bromide pellets.

ii. X-ray Powder Diffraction (XRD)

The X-ray diffractograms were obtained using a RX SIEMENS D5000 diffractometer with a vertical goniometer and a copper anodic tube, radiation CuKα, λ=1.54056 Å.

iii. Differential Scanning Calorimetry (DSC)

DSC measurements were carried out in vented pan at a scan rate of 10° C./minute from 25.0° C. to 180.0° C. under a nitrogen purge with a Mettler-Toledo DSC821.

iv. Thermogravimetric Analysis (TG)

TG measurements were carried out in a vented pan at a scan rate of 10° C./minute from 25.0° C. to 180.0° C. under a nitrogen purge with a Mettler-Toledo TG50 thermobalance.

V. Particle Size Measurement

Particle size measurements were obtained using a Coulter LS particle size analyzer. Soy lecithin (0.5 g) was dissolved in 20 mL of hexane at room temperature and poured (˜15 mL) into a measurement cell. Solid samples were added to approximately 5 mL of the soy lecithin solution and ultrasonicated for one minute. The sonicated solution was then added dropwise to the measurement cell until the correct obscuration was obtained, typically 8-12%. Measurements are reported in μm.

For Examples 6 and 6A, particle size was measured using a Malvern Mastersizer S particle size analyzer with an MS1 Small Volume Recirculating unit attached. A 300RF mm lens and a beam length of 2.4 mm was used. Samples for analysis were prepared by dispersing a weighed amount of aripiprazole (approximately 0.5 g) in 100 mL of soybean oil in hexane 0.5%. The suspension was sonicated for 5 minutes and delivered drop-wise to a background corrected measuring cell previously filled with soybean oil in hexane (0.5%) until the obscuration reached the desired level. Volume distributions were obtained for three times. Upon measurement completion, the sample cell was emptied, cleaned and refilled with suspending medium. The sampling procedure was then repeated. For characterization, the values of D10, D50 and D90 were specifically listed, each one being the mean of the six values available for each characterization parameter.

vi. HPLC Method

Chromatographic separation was carried out using a Luna C18(2), 5 μm. 25 cm×4.6 mm. I.D column.

The mobile phase A was prepared by mixing 370 mL of acetonitrile with 630 mL of buffer (pH=4.0) prepared from 0.7 g KH₂PO₄ and 1.2 g of 1-pentanesulfonic acid sodium salt dissolved in 630 mL of water. The pH was adjusted to 4.0 by the addition of 10% orthophosphoric acid. The mobile phase was mixed and filtered through a 0.22 μm nylon filter under vacuum.

The mobile phase B is acetonitrile.

The chromatograph was programmed as follows: 0-34 minutes isocratic 100% mobile phase A, 34-50 minutes linear gradient to 75% mobile phase A, 50-70 minutes isocratic 75% mobile phase A, 70-75 minutes linear gradient to 100% mobile phase A and 75-80 minutes equilibration with 100% mobile phase A.

The chromatograph was equipped with a 215 nm detector and the flow rate was 1.2 mL per minute. Chromatographic runs were performed at room temperature. Test samples (20 μL) were prepared by dissolving the appropriate amount of sample in order to obtain 1 mg per mL in a 6:4 mixture of acetonitrile/water (v/v).

vii. Hygroscopicity Measurements

One gram of the sample was accurately weighed in a weighing bottle (diameter 5 cm), covered with kimwipes and left to rest in 60° C./100% RH environment (water/dessicator). 24 hours later, the weighing bottle was removed, transferred to an environment of room temperature and about 30% RH (magnesium chloride hexahydrate saturated water solution/dessicator) and left to rest for 24 hours and the water content of the sample was measured by the Karl Fisher method.

viii. Gas Chromatography

Chromatographic separation was carried out in a TRB-624 capillary column of 1.8 μm film thickness, 75 m×0.53 mm i.d. column. The chromatograph was equipped with a FID detector and a Head Space injection auxiliary device.

The oven temperature is programmed as follows: Initial 0-20 minutes 50° C., then the temperature was raised to 225° C. (ramp rate 5°/minute) and was maintained at 225° C. for 5 minutes. The injector and detector temperatures were set at 225° C. and 250° C. respectively. Helium was used as carrier gas at a pressure of 7 psi with a split. Samples were heated for 45 minutes at 80° C. in the head space device. After heating, the vials were pressurized with helium at 18 psi for 0.2 minutes. The sample loop was filled for 0.2 minutes (loop volume=3 mL) and then injected for 1 minute.

Solutions:

Standard Ethanol Solution (100 ppm): Dilute quantitatively 100 mg of Ethanol with 100 mL of dimethyl sulfoxide and dilute 1 mL of this solution to 10 mL with dimethyl sulfoxide to obtain a solution containing 0.01 μg/mL.

Test solution: Prepare a solution of about 100 mg. of Aripiprazole test sample in 5 mL of dimethyl sulfoxide.

Procedure: The vials were sealed with suitable crimp caps and analyzed by headspace using the above-described conditions. A blank run was performed using dimethylsulfoxide and then disregarding the peaks corresponding thereto in the test and standard solution runs.

ix. Specific Surface

The BET (Brunauer, Emmett and Teller) specific surface for aripiprazole was measured using a Micromeritics ASAP2010 equipment. Samples for analysis were degassed at 100° C. under vacuum for two hours. The determinations of the adsorption of N₂ at 77° K were measured for relative pressures in the range of 0.07-0.2 for a weighed amount of 400 mg.

x. Single Crystal X-ray Analysis

X-ray data for single crystal of aripiprazole Form J was collected at 294(2) K on an Enraf-Nonius CAD 4 diffractometer using Mo—K_{α} radiation.

### Reference Example 1

### Aripiprazole Form H

This example illustrates the preparation of aripiprazole according to Example 1 of U.S. Pat. No. 5,006,528.

In a suitable reactor, 23.50 g (78.8 mmol) of 7-(4-bromobutoxy)-3,4-dihydro-2(1M-quinolinone, 17.50 g (117 mmol) of sodium iodide are combined in 250 mL of acetonitrile. The resulting suspension was stirred and heated to reflux temperature (˜80° C.). After stirring for approximately 30 minutes at reflux temperature, 20.00 g (86.5 mmol) of 1-(2,3-dichlorophenyl)piperazine, 16.5 mL (119 mmol) of triethylamine and 50 mL of acetonitrile were added to the reactor via an addition funnel. The mixture was then stirred at reflux for approximately three hours. After cooling, the solvent was removed by rotary evaporation to yield a yellowish solid.

The recovered solid was dissolved in a combined 200 mL of chloroform and 200 mL of water to give an orange organic phase and a colorless aqueous phase. The organic phase was separated and re-extracted with 200 mL of water. After drying overnight over anhydrous sodium sulphate, the mixture was filtered to give a clear orange solution. The solvent was then removed by rotary evaporation to give a yellow solid.

The recovered solid was recrystallized by heating in 300 mL of ethanol, cooling the solution to approximately 0-5° C. and stirring at this temperature for at least 30 minutes. The mixture was then filtered and the collected solid was washed with 20 mL of ethanol to yield a white, crystalline solid. The resulting solid was then recrystallized again by heating in 250 mL of ethanol and cooling the solution to approximately 0-5° C. with stirring at this temperature for at least 30 minutes. The mixture was then filtered and the collected solid was washed with 20 mL of ethanol to yield a white, crystalline solid. The solid was dried under vacuum at room temperature to give 81.4% of crystalline aripiprazole.

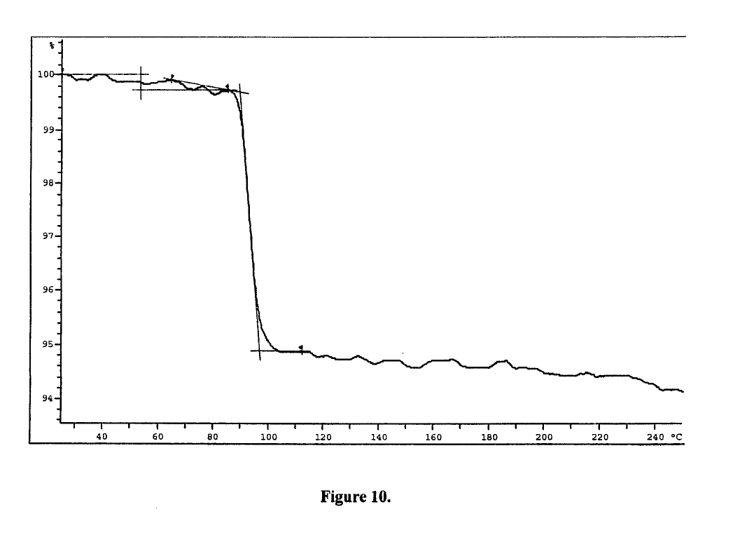

Analytical data: Assay: 95.12%; Melting point (NP): 139.0-139.4° C.; DSC (open pan): endothermic peaks at about 98.8° C. and 140° C., see FIG. 6; DSC (sealed pan): endothermic peaks at about 98.5° C. and 133.3° C., see FIG. 7; TG: A loss of weight of 4.8% is observed between about 89.7° C. and 93° C., see FIG. 10; XRD (2θ): 8.8°, 10.2°, 12.6°, 17.5°, 18.0°, 18.2°, 19.7°, 23.3°, 24.5°, 27.9°; see FIG. 2; IR: see FIG. 12.

Notably, the aripiprazole obtained in Reference Example 1, and Comparative Examples 1A and 1B exhibits an XRD substantially identical to that of FIG. 2 and contains a certain amount of residual ethanol. This result is in accordance with the assay result (˜95%) and TG loss of weight (˜4.8%). The theoretical content of ethanol for aripiprazole monoethanolate, however, is 9.32% (w/w). Therefore, the aripiprazole obtained in Reference Example 1 (as well as Comparative Examples 1A and 1B) is a crystalline hemiethanolate form of aripiprazole (i.e., aripiprazole hemiethanolate), herein designated Form H and exhibiting an XRD substantially identical to that of FIG. 2.

Three portions of the crystals obtained in Reference Example 1 were dried for 6 additional hours at three different temperatures under vacuum and re-analyzed. The drying conditions and the characteristics of the re-analyzed samples are reported in Comparative Examples 1A, 1B and 1C.

### Comparative Example 1A

### Aripiprazole Form H

A sample of the crystals obtained in Reference Example 1 was dried under vacuum at approximately 60° C. for 6 hours. The loss of mass during drying was 1%.

Analytical data: Assay: 95.35%; MP: 138.8-139.8° C.; DSC (open pan and sealed pan) are substantially identical to FIGS. 6 and 7, respectively; TG: substantially identical to FIG. 10; XRD (2θ): 8.7°, 10.2°, 12.5°, 17.4°, 18.1°, 19.6°, 23.3°, 24.5°, 25.3°, 27.8°, substantially identical to FIG. 2; IR: substantially identical to FIG. 12.

### Comparative Example 1B

### Aripiprazole Form H

A sample of the crystals obtained in Reference Example 1 was dried under vacuum at approximately 40° C. for 6 hours. The loss of mass during drying was 0%.

Analytical data: Assay: 95.03%; MP: 139.0-139.4° C.; DSC (open pan and sealed pan) are substantially identical to FIGS. 6 and 7, respectively; TG: substantially identical to FIG. 10; MD (2θ): 8.7°, 10.2°, 12.6°, 17.4°, 17.9°, 18.1°, 19.7°, 23.3°, 24.5°, 27.9°, substantially identical to FIG. 2; IR: substantially identical to FIG. 12.

### Comparative Example 1C

### Aripiprazole Form L

A sample of the crystals obtained in Reference Example 1 was dried under vacuum at approximately 90° C. for 6 hours. The loss of mass during drying was 5%.

Analytical data: HPLC purity: 99.93%; Assay: 99.99%; MP: 138.8-139.3° C.; DSC (open pan): endothermic peak at about 139.3° C., see FIG. 8; XRD (2θ): 11.0°, 12.1°, 14.4°, 14.9°, 16.6°, 17.0°, 19.3°, 19.5°, 20.4°, 22.1°, 26.6°, 27.1°, 28.3°, see FIG. 3; IR: see FIG. 13.

### Reference Example 2

### Aripiprazole Form K

Reference Example 2 illustrates the preparation of aripiprazole by recrystallization from ethyl acetate.

In a suitable reactor, 50.00 g of 7-(4-bromobutoxy)-3,4-dihydro-2(1H)-quinolinone, 45.77 g of 1-(2,3-dichlorophenyl)piperazine hydrochloride, 25.64 g of sodium iodide, 250 mL of acetonitrile and 35.63 g of triethylamine were combined. The resulting suspension was stirred and heated to reflux temperature (˜82° C.) and maintained at this temperature for approximately 3 hours. The suspension was cooled to approximately 0-5° C. and stirred at this temperature for approximately 1 hour. The suspension was then filtered and the resulting solid was washed with 34.7 mL of acetonitrile. This crude material has an HPLC purity of 95.94%.

The wet solid was next placed in a suitable reactor with 537.95 mL of water. The resulting suspension was stirred and heated to reflux temperature (˜100° C.) and allowed to cool. The suspension was then filtered at approximately 40-45° C. and the isolated solid was washed with 25.57 mL of water. This crude material has an HPLC purity of 98.58%.

The isolated wet solid from the previous step was then placed in a suitable reactor with 481.18 mL of acetone. The stirred suspension was heated to reflux temperature (˜54-56° C.) and maintained at this temperature for at least 30 minutes. The suspension was then cooled to approximately 10-15° C. and stirred at this temperature for approximately 1 hour. The suspension was then filtered and the isolated solid was washed with 14.54 mL of acetone. This material has an HPLC purity of 99.59%.

The wet solid from the previous step was then placed in a suitable reactor with 465.97 mL of acetone. The suspension was stirred and heated to reflux temperature (˜54-56° C.) and maintained at this temperature for approximately 30 minutes. The suspension was then cooled to approximately 10-15° C. and stirred at this temperature for approximately 1 hour. The suspension was filtered and the isolated solid was washed with 14.14 mL of acetone to yield crude aripiprazole wet of acetone (yield 83.3%). This material has an HPLC purity of 99.82%. X-ray powder diffraction of this material indicates it is a mixture of different polymorphic forms of aripiprazole.

The wet aripiprazole (estimated dry mass: 62.50 g) was combined with 750.00 mL of ethyl acetate in a suitable reactor. The suspension was stirred and heated to reflux temperature (˜78° C.) and maintained at this temperature until dissolution occurs. The solution was then filtered at approximately 64° C. through a filter aid (Avicel® PH-101) and the filter aid was further rinsed with 11.97 mL of ethyl acetate. The filtrate was re-heated to reflux and maintained at this temperature until complete dissolution occurred. The resulting mixture was cooled to approximately 0-5° C. and stirred at this temperature for at least 1 hour. The suspension was filtered and the collected solid was washed with 35.91 mL of ethyl acetate. The wet product was dried under vacuum at approximately 60° C. to constant mass, to yield 54.32 g (86.9%) of crystalline aripiprazole.

Analytical data: HPLC purity: 99.79%; Assay: 100.4%; MP: 148.8-149.5° C.; DSC (open pan): two endothermic peaks at 139.85° C. and 148.98° C., see FIG. 9; XRD (2θ): 14.4°, 16.6°, 19.4°, 20.5°, 22.1°, 22.8°, 26.6°, see FIG. 4; IR: substantially identical to FIG. 13; Particle size: 29.9 μm (mean); Residual solvents: <100 ppm acetonitrile, <100 ppm acetone, 448 ppm ethyl acetate; Water content (Karl Fisher method): 0.08%; Hygroscopicity: 0.35%.

### Example 1

### Aripiprazole Form J

This example illustrates the preparation of aripiprazole Form J produced by recrystallization of crude aripiprazole (wet from acetone) from methyl ethyl ketone.

Crude aripiprazole wet from acetone was obtained according to the description of Reference Example 2 (above). The wet aripiprazole was recrystallized from methyl ethyl ketone instead of ethyl acetate (as described in Reference Example 2).

Wet aripiprazole (estimated dry mass: 17.75 g) was combined with 142.00 mL of methyl ethyl ketone in a suitable reactor. The stirred suspension was heated to reflux temperature (˜78° C.) and maintained at this temperature until dissolution occurred. The solution was then cooled and filtered at approximately 65° C. through a filter aid (Avicel® PH-101) and the filter aid was rinsed with an additional 5 mL of methyl ethyl ketone. The filtrate was re-heated to reflux and maintained at this temperature until complete dissolution occurs. The resulting mixture was cooled to approximately 0-5° C. and stirred at this temperature for at least 1 hour. The suspension was filtered and the isolated solid was washed with 17.75 mL of methyl ethyl ketone. The wet product was dried under vacuum at approximately 65° C. to constant mass to yield 15.92 g (89.7%) of aripiprazole Form J.

Analytical data: HPLC purity of 99.84%; Assay: 99.36%; MP: 147.5-148.3° C.; DSC (open pan): two endothermic peaks at 120.66° C. and 148.90° C., see FIG. 5; XRD (2θ): 5.4°, 10.0°, 10.8°, 11.6°, 12.6°, 15.7°, 15.9°, 16.3°, 18.5°, 19.8°, 20.0°, 20.5°, 21.8°, 22.3°, 23.1°, 23.3°, 24.5°, 26.0°, 27.1°, 28.8°, see FIG. 1; IR: see FIG. 11; Particle size: 22.7 μm (mean); Water content (Karl Fisher method): 0.11%; Hygroscopicity: 0.06%; Specific surface area: 0.7447±0.0317 m²/g.

### Example 2

### Aripiprazole Form J

This example illustrates the preparation of aripiprazole Form J produced by recrystallization of crude aripiprazole from methyl ethyl ketone.

Aripiprazole (15.00 g) and 120 mL of methyl ethyl ketone were combined in a suitable reactor. The suspension was stirred and heated to reflux (˜80° C.). The suspension was then cooled to approximately 0-10° C. with continued stirring. The suspension was filtered and the isolated solid was washed with approximately 10 mL of methyl ethyl ketone. The wet product was dried under vacuum at approximately 60° C. to a constant mass to yield 12.75 g (85.0%) of aripiprazole Form J.

Analytical data: HPLC purity of 99.91%; Assay: 99.71%; MP: 148.2-149.1° C.; DSC (open pan): two endothermic peaks at 120.66° C. and 149.21° C., substantially identical to FIG. 5; XRD (2θ): 5.4°, 10.0°, 10.8°, 11.6°, 12.6°, 15.7°, 15.9°, 16.3°, 18.6°, 19.5°, 19.8°, 20.0°, 20.5°, 21.8°, 22.3°, 23.3°, 24.5°, 26.0°, 27.1°, 28.4°, 28.8°, 29.5°, 32.6°, 33.7°, 36.7°, substantially identical to FIG. 1; IR: substantially identical to FIG. 11; Residual solvents: 587 ppm methyl ethyl ketone.

### Example 3

### Aripiprazole Form J

This example illustrates the preparation of aripiprazole Form J produced by recrystallization of crude aripiprazole from methyl ethyl ketone.

Dry aripiprazole (19.8 Kg) and 80 Kg of methyl ethyl ketone were combined in a suitable reactor. The resulting suspension was stirred and heated to reflux temperature (˜78° C.) and maintained at this temperature until dissolution occurred. The solution was cooled to approximately 0-5° C. and maintained at this temperature for at least 2 hours. The suspension was then filtered and the isolated solid was washed with approximately 5 Kg of methyl ethyl ketone. The resulting solid was dried under vacuum at approximately 60±5° C. to constant mass, sieved and blended to obtain 17.64 Kg (89.1%) of aripiprazole Form J.

Analytical data: HPLC purity: 99.91%; Assay: 100.65%; MP: 148.5-149.3° C.; DSC (open pan): endothermic peaks at 116.65° C. and at 149.51° C.; substantially identical to FIG. 5; XRD (2θ): 5.4°, 10.0°, 10.8°, 11.6°, 15.7°, 16.3°, 18.6°, 19.5°, 19.8°, 20.5°, 21.8°, 22.3°, 23.4°, 24.5°, 26.1°, 27.1°, 28.4°, 28.8°, 29.5°, 32.6°, 33.7°, 38.3°, 45.4°, 45.5°, substantially identical to FIG. 1; IR: substantially identical to FIG. 11; Particle size: 27 μm (mean).

### Example 4

### Aripiprazole Form J

This example illustrates the preparation of aripiprazole Form J produced by acetone digestion.

Aripiprazole (0.5 g) was suspended in 7.5 mL of acetone and heated to reflux (˜56° C.) for approximately 1 hour. The suspension was then slowly cooled to room temperature. The resulting wet crystalline aripiprazole Form J was collected by filtration. The isolated aripiprazole Form J was dried under vacuum at approximately 70° C. for 15 hours to yield 0.39 g (78%) of product.

Analytical data: HPLC purity: 99.91%; Assay: 100.2%; MP: 148.4-149.1° C.; DSC (open pan): endothermic peaks at 121.97° and 148.79° C.; substantially identical to FIG. 5; XRD (2θ): 5.4°, 10.0°, 10.8°, 11.6°, 12.6°, 15.7°, 16.2°, 18.5°, 19.8°, 20.4°, 21.8°, 22.2°, 23.3°, 24.4°, 26.0°, 27.1°, 28.7°; substantially identical to FIG. 1; IR: substantially identical to FIG. 11.

### Example 5

### Aripiprazole Form J

This example illustrates the preparation of aripiprazole Form J produced by acetone digestion.

Aripiprazole (0.5 g) was suspended in 7.5 mL of acetone and heated to reflux (˜56° C.) for approximately 1 hour. The suspension was then slowly cooled to room temperature over 1.5 hours. The resulting wet crystalline aripiprazole Form J was collected by filtration. The isolated aripiprazole Form J was dried under vacuum at approximately 70° C. for 4 hours to yield 0.3 g (60%) of product.

Analytical data: HPLC purity: 99.9%; Assay: 100.05%; MP: 148.2-149.0° C.; DSC (open pan): substantially identical to the DSC shown in FIG. 5; XRD (2θ): 5.3°, 9.9°, 10.7°, 11.5°, 15.6°, 16.2°, 18.5°, 19.8°, 20.0°, 20.4°, 21.7°, 22.2°, 23.3°, 26.0°, 27.0°, 28.7°, substantially identical to FIG. 1; IR: substantially identical to FIG. 11.

### Reference Example 6

### Aripiprazole Form H

This example illustrates the preparation of aripiprazole Form H by recrystallization from ethanol and drying the resulting product.

Dry aripiprazole (10 Kg) and 105 Kg of ethanol were combined in a suitable reactor. The resulting mixture was stirred and heated to reflux temperature (˜79° C.) and maintained at this temperature until dissolution occurred. The hot solution was filtered at a temperature above 70° C., cooled to approximately 0-5° C., and maintained at this temperature for at least 2 hours. The suspension was then filtered and the isolated solid was washed with approximately 4 Kg of ethanol.

The resulting wet solid (9.6 Kg) and 84 Kg of ethanol were combined again in a suitable reactor. The suspension was stirred and heated to reflux temperature (˜79° C.) and maintained at this temperature until dissolution occurred. The solution was then cooled to approximately 0-5° C. and maintained at this temperature for at least 2 hours. The suspension was then filtered and the isolated solid was washed with approximately 4 Kg of ethanol. The resulting wet product was dried under vacuum at 60° C. over 6 hours to yield 9.45 Kg of crystalline aripiprazole (i.e., equivalent to the product of Reference Example 1 and Comparative Examples, 1A and 1B).

Analytical data: HPLC purity: 99.8%; Assay: 95.18%; XRD (2θ): substantially identical to FIG. 2;); Residual Solvent (ethanol) (by Head Space Gas Chromatography): 5.11%; IR: substantially identical to that of FIG. 12; TG: substantially identical to FIG. 10; Particle size distribution (volume): 10% of the particles have a diameter below 15.83 μm, 50% of the particles have a diameter below 45.64 μm and 90% of the particles have a diameter below 85.62 μm.

### Comparative Example 6A

### Mixture of Aripiprazole Form L and Form H

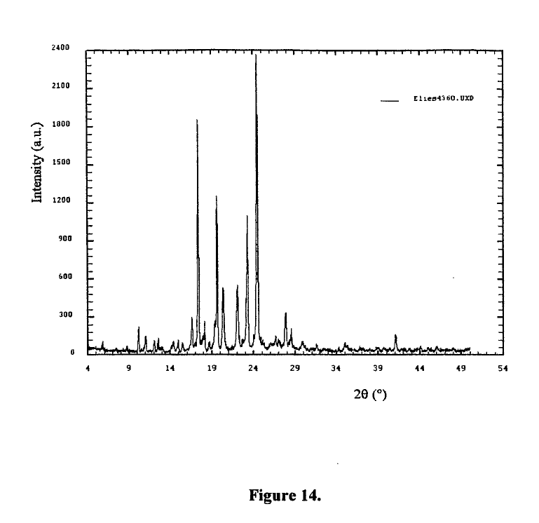

40 g of aripiprazole obtained in Reference Example 6 was dried at 80° C. for 15 hours to yield 38.83 g of crystalline aripiprazole. The crystalline aripiprazole obtained exhibits the XRD spectrum illustrated in FIG. 14, corresponding to a mixture of aripiprazole Form H and Form L.

Analytical data: HPLC purity: 99.8%; Assay: 97.86%; MP: 138.3-139.1° C.; Residual solvent (ethanol): 3.08%; DSC (open pan): three endothermic peaks (onset at 94.16° C., 138.82° C. and 143.70° C.); XRD (2θ): 5.8°, 10.2°, 11.0°, 12.1°, 12.5°, 14.1°, 14.4°, 15.0°, 15.4°, 16.6°, 17.3°, 18.1°, 18.7°, 19.4°, 19.6°, 20.4°, 22.1°, 23.1°, 23.3°, 24.0°, 24.4°, 24.8°, 26.7°, 27.8°, 28.5°, 41.1°, see FIG. 14. Particle size distribution (volume): 10% of the particles have a diameter below 16.1 μm, 50% of the particles have a diameter below 43.9 μm and 90% of the particles have a diameter below 76.9 μm.

What is claimed is:

1. Aripiprazole crystalline Form J characterized by an X-ray powder diffraction pattern (2θ) (±0.2°) having its main peaks at approximately 15.64°, 15.90°, 16.23°, 21.77°, 22.22° and 23.27°, wherein said aripiprazole has a mean particle size of approximately 100 μm or less.

2. The aripiprazole crystalline Form J according to claim 1 further characterized by an X-ray powder diffraction pattern (2θ) (±0.2°) having peaks at approximately 5.33°, 9.93°, 10.71°, 11.55°, 12.55°, 18.49°, 18.89°, 19.45°, 19.75°, 19.99°, 20.42°, 24.43°, 25.97°, 27.04°, 28.36°, 28.73°, 29.42° and 33.61°, wherein said aripiprazole has a mean particle size of approximately 100 μm or less.

3. The aripiprazole crystalline Form J of claim 1, wherein said aripiprazole has a mean particle size of approximately 50 μm or less.

4. The aripiprazole crystalline Form J of claim 3, wherein said aripiprazole has a mean particle size of approximately 30 μm or less.

5. The aripiprazole crystalline Form J of claim 1, wherein said aripiprazole has a specific surface area of approximately 0.7447 m²/g.

6. The aripiprazole crystalline Form J of claim 1 wherein said aripiprazole has a purity of at least 99.8% by HPLC.

7. The aripiprazole crystalline Form J of claim 6 wherein said aripiprazole has a purity of at least 99.9% by HPLC.

8. A pharmaceutical composition comprising at least one of said aripiprazole crystalline Form J of claim 1 and a pharmaceutically acceptable salt thereof.

9. The pharmaceutical composition of claim 8, further comprising at least one of a an adjuvant, a carrier, a diluent and another crystalline form of aripiprazole or a pharmaceutically acceptable salt thereof.

10-13. (canceled)

13. A process for preparing aripiprazole crystalline Form J comprising recrystallizing at least one other form of aripiprazole in a ketonic solvent.

14. The process of claim 13, wherein said ketonic solvent is at least one of methyl ethyl ketone, acetone and mixtures thereof.

15-24. (canceled)

25. The pharmaceutical composition of claim 8, wherein said aripiprazole crystalline Form J or pharmaceutically acceptable salt thereof has a purity of at least 99.8% by HPLC.

26. The pharmaceutical composition of claim 8, wherein said aripiprazole crystalline Form J or pharmaceutically acceptable salt thereof has a purity of at least 99.9% by HPLC.

27. A process for preparing aripiprazole crystalline Form L comprising converting an aripiprazole solvate into aripiprazole crystalline Form L.

28. The process of claim 27, wherein said aripiprazole solvate is at least one of a methanolate, an ethanolate, a butanolate and a propanolate.

29. The process of claim 27, wherein said aripiprazole solvate is a hemisolvate.

30. The process of claim 29, wherein said aripiprazole solvate is a hemiethanolate.

31. The process of claim 27, further comprising drying at least one aripiprazole solvate at a temperature between approximately 60° C. and approximately 120° C.

32. The process of claim 31, wherein said temperature is between approximately 90° C. and approximately 110° C.

33. The process of claim 32, wherein said temperature is approximately 100° C.

34. Aripiprazole crystalline Form L made according to the process of claim 27, wherein said aripiprazole crystalline Form L has a purity of at least 99.8% by HPLC.

35. Aripiprazole crystalline Form L made according to the process of claims 27, wherein said aripiprazole crystalline Form L has a purity of at least 99.9% by HPLC.

36. A pharmaceutical composition comprising at least one of the aripiprazole crystalline Form L of claim 34 and a pharmaceutically acceptable salt thereof.

37. The pharmaceutical composition of claim 36, further comprising at least one of a an adjuvant, a carrier, a diluent and another crystalline form of aripiprazole or a pharmaceutically acceptable salt thereof.

38-45. (canceled)
